(19) United States
(12) Patent Application Publication
(10) Pub. No.: US 2008/0085256 A1
(43) Pub. Date: Apr. 10, 2008

(54) Vitality Restoring Composition Containing DHEA and Minoxidil

(76) Inventor: Doanh Manh Nguyen, Long Beach, CA (US)

Correspondence Address: ORION LAW GROUP, 3 HUTTON CENTRE, SUITE 850, SANTA ANA, CA, 92707, US

(21) Appl. No.: 11/539,550

(22) Filed: Oct. 6, 2006

(51) Int. Cl. A61K 8/49; A61K 8/36

(52) U.S. Cl. 424/70.22; 514/269; 514/560

(57) ABSTRACT

The present invention relates to a composition that reduces age related symptoms and provides increased energy and activity levels.

### BACKGROUND OF THE INVENTION

The present invention relates to a composition comprising DHEA and MINOXIDIL for purposes of providing energy and minimizing numerous conditions related to aging.

DHEA is a natural steroid produced by the body by the corticoadrenal glands. Prior art uses of synthetic DHEA or dehydroepiandrosterone have largely addressed cosmetic uses, such as reducing the appearance of wrinkles, minimizing fine lines, and improving the appearance of sun damaged epidermis (U.S. Pat. No. 6,852,326). DHEA has also been used to promote the keratinization of the epidermis (JP-07 196 467).

Additional uses of DHEA include the reduction of body fat and increased muscle mass (U.S. Pat. No. 4,920,115), improvement of prostate hypertrophy and sexual dysfunction (U.S. Pat. No. 4,835,147), and reduced morbidity and mortality from vascular diseases (U.S. Pat. No. 5,110,810).

Minoxidil is a vasodilator and was originally developed to treat hypertension. Physicians treating patients for hypertension with minoxidil observed an interesting side effect of increased hair growth. From these observations, Minoxidil has since been commonly used to address male pattern baldness and promote hair growth through topical applications (U.S. Pat. No. 6,596,266). Thus, prior art uses of minoxidil are related to cardiovascular conditions and cosmetic purposes.

The present invention comprises a composition containing DHEA and minoxidil. When the composition is applied to target areas as a topical application conditions related to aging are reduced. The composition may be used as a youth-promoting elixir. Symptoms that the composition may improve or reduce include fatigue, constipation, joint and soft tissue pain, gastro-esophageal reflux, hair loss, and nocturnal voiding. Users may also experience psychological benefits such as increased energy levels and improved optimism.

### SUMMARY OF THE INVENTION

The present invention is a composition containing DHEA and minoxidil applied topically to reduce and minimize symptoms related to aging. The composition is provided in a physiologically acceptable medium and in various formats including gel, cream, lotion, or liquid form.

The composition may be applied on the scalp or on the legs of the user. Locations with prominent veins may be an effective location to administer the composition and may allow for more rapid absorption of the composition. The present invention provides physiologically-active ingredients at low dosages for safe and effective use by the user.

It is an object of the present invention to provide a composition that reduces muscle and joint pain.

It is another object of the present invention to provide a composition that reduces fatigue and increases energy levels.

It is an additional object of the present invention to provide a composition that promotes regular bowel movements.

It is a further object of the present invention to provide a topical application that supplies safe and effective dosages of DHEA and minoxidil for purposes of providing youth related benefits.

It is an additional object of the present invention to provide a composition that can be delivered effectively via topical applications.

### DETAILED DESCRIPTION OF PREFERRED EMBODIMENTS

The present invention provides a composition containing DHEA and minoxidil. The applicant has discovered the non-obvious outcome that when the composition is administered topically, a user feels energized and many conditions related to aging are minimized. Applicant's theory on the mechanism of the present invention is based on the ability of the composition to act as a catalyst and stimulate the transcription of essential genes that are transcribed at higher levels in younger populations.

The composition allows for the unlocking of DNA binding domains resulting in the transcription of essential genes. The transcription of essential genes allows for the regulation of protein synthesis. Applicant hypothesizes that the transcription of genes regulated by endogenous DHEA occurs more frequently in younger populations and hence a relationship exists between said proteins and qualities related to youth. By stimulating the transcription of said genes, youthfulness may be mimicked. The pathway may be simplified as follows:

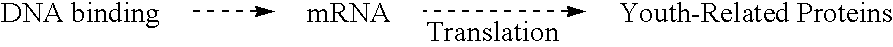

The basis for applicant's hypothesis stems from the finding that DHEA levels in humans decline with the advancement of age. Serum DHEA and DHEA-S levels peak in humans at around 25 years of age and begin to decline after age 30. Aged populations will possess 5% of their original DHEA levels and individuals suffering from chronic illnesses such as type 2 diabetes may also have lower DHEA levels. Thus there appears to be a relationship between youth, healthy immune systems, and DHEA levels in the human body.

It is applicant's hypothesis that by introducing low levels of DHEA and/or minoxidil at certain sites in a user's body, up-regulation of key receptors related to youth will be stimulated. The transcription of youth related genes and translation of youth related proteins will occur at higher levels due to stimulation from the composition. Because the composition serves as a catalyst, introduction of only miniscule levels are needed to produce the desired effect. The low dosages of active ingredients further prevent bioaccumulation of DHEA or minoxidil from occurring.

Applicant has observed that when a DHEA solution is applied topically in a concentration range of 0.2% to 0.5% numerous symptoms and conditions related to aging are reduced. In the preferred embodiment of the present invention however, the applicant has found that the non-obvious combination of DHEA combined with minoxidil provides a more potent, vitality-producing effect than the DHEA solution alone.

In the preferred embodiment of the present invention, the DHEA should have a concentration within the range of 0.2% to 0.5%. The minoxidil may be in the range of 2% to 5%. The preferred embodiment of the composition is in a liquid form, however numerous mediums may be used to deliver the active ingredients. Gels, lotions, pastes, mousses, or patches are all suitable mediums for absorption of the composition. Such mediums may be provided by means and methods known to persons skilled in the art.

To prepare a solution of the present invention 1.2 gm of minoxidil USP powder may be combined with 0.12 gm of DHEA USP powder. 45.6 ml. of 95% Ethanol, 10.8 ml. of purified water, and 3.6 ml of propylene glycol may be combined and added to the solid mixture. The solution should be stirred until the solute is completely dissolved. The resulting composition is one embodiment of the present invention and may be applied directly on the epidermis of the user.

For preparation of a gel format of the present invention the following steps may be carried out: using a mortar and pestle, 1.2 gm of minoxidil USP powder is mixed with a few drops of 95% EtOH until the solid is dissolved. The resulting solution may be mixed with a portion of a transderma base such as PLO Ultramax gel to form a smooth nucleus. Incorporation of the balance of the transderma base is carried out by geometric dilution until the final weight of the composition is 30 gm. The combination is mixed until the gel has reformed into an even, smooth, and silky consistency. The above steps are repeated, replacing the minoxidil with 0.12 gm of DHEA USP powder. The two gels are thoroughly mixed together until uniform and the total final weight of the composition should weigh 60 gm. The final composition may be applied directly to the epidermis.

Variations of the above may be carried out, while still falling within the spirit of the invention. The following tables illustrate additional examples of formulations of the present invention. The examples are not exhaustive however, and additional embodiments of the invention may exist.

### EXAMPLE 1

Topical Solution

| | | |
|---|---|---|
| Minoxidil USP Powder | 1.2 | gm |
| DHEA USP powder | 0.12 | gm |
| 95% EtOH | 45.6 | ml |
| Purified water | 10.8 | ml |
| Propylene glycol | 3.6 | ml |

### EXAMPLE 2

Topical Solution

| | | |
|---|---|---|
| Minoxidil USP Powder | 1.2 | gm |
| DHEA USP powder | 0.12 | gm |
| Isopropyl alcohol | 45.6 | ml |
| Purified water | 10.8 | ml |
| Propylene glycol | 3.6 | ml |

### EXAMPLE 3

Topical Solution

| | | |
|---|---|---|
| Minoxidil USP Powder | 1.2 | gm |
| DHEA USP powder | 0.12 | gm |
| Isopropyl alcohol | 45.6 | ml |
| Purified water | 10.8 | ml |

### EXAMPLE 4

Topical Solution

| | | |
|---|---|---|
| Minoxidil USP Powder | 1.2 | gm |
| DHEA USP powder | 0.12 | gm |
| SD alcohol | 45.6 | ml |
| Purified water | 10.8 | ml |

### EXAMPLE 5

Topical Solution

| | | |
|---|---|---|
| Minoxidil USP Powder | 1.2 | gm |
| DHEA USP powder | 0.12 | gm |
| SD alcohol | 45.6 | ml |
| Purified water | 10.8 | ml |
| Propylene glycol | 3.6 | ml |

### EXAMPLE 6

Topical Gel

| | |
|---|---|
| Minoxidil USP Powder | 1.2 gm |
| DHEA USP powder | 0.12 gm |
| PLO Ultramax Gel | 58.6 gm |
| 95% EtOH | a few drops |

Application of the solution form of the composition may be accomplished with conventional cotton swabs or cotton pads. A typical volume required for sufficient absorption may be 1 ml. This value, however, may vary depending on the sensitivity of the user. The user simply absorbs solution onto the swab or pad and directly applies to the application site daily. Users may see results in as soon as 1 week.

Applicant has noted that some users may experience irritation if the composition is applied to the scalp area. An alternative area may be over prominent veins on the arms or legs. Varicose veins on the legs have also been shown to be an effective site for application of the composition.

In summary, the present invention is a novel and inventive composition and process for increasing the production of youth-related proteins allowing for the mimicking of youthfulness. With respect to the above description, it is to be realized that variations are deemed readily apparent and obvious to one skilled in the art, and all equivalent relationships to those illustrated in the tables and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact composition and operation shown and described, and accordingly, all suitable modifications and equivalents may be resorted to, while still falling within the scope of the invention.

1. A composition comprising:
A physiologically acceptable medium for topical application;
Minoxidil; and
DHEA.

2. The composition as claimed in claim 1, comprising:
a Minoxidil solution having a concentration of 2%-3%.

3. The composition as claimed in claim 1, comprising:
A DHEA solution having a concentration of 0.2-0.5%.

4. The composition as claimed in claim 2 comprising a DHEA solution having a concentration of 0.2-0.5%.

5. The composition as claimed in claim 1 whereby said medium is in the form of Carbomer 940.

6. The composition as claimed in claim 1 whereby said medium is in the form of disodium lauryl sulfosuccinate.

7. The composition as claimed in claim 1 whereby said medium is in the form of dioctyl sodium sulfosuccinate.

8. A process comprising:
applying the composition of claim 1 for purposes of stimulating transcription of genes stimulated by endogenous DHEA.

9. A process comprising:
applying the composition of claim 1 for purposes of minimizing age-related symptoms.

10. A process comprising:
applying the composition of claim 1 topically on a human in an amount sufficient to provide increased energy and activity levels.

11. A process comprising:
applying the composition of claim 1 topically on a human in an amount sufficient to reduce strained vision.

12. A process comprising:
applying the composition of claim 1 topically on a human in an amount sufficient to minimize age related symptoms affecting urination including minimizing urgency and frequency of urination as well as difficulty in initiating flow of urination.

13. A process comprising:
applying the composition of claim 1 topically on a human in an amount sufficient to reduce gastro-esophageal reflux.

14. A process comprising:
applying the composition of claim 1 topically on a human in an amount sufficient to minimize fatigue.

15. A process comprising:
applying the composition of claim 1 topically on a human in an amount sufficient to reduce muscle pain.

16. A process comprising:
applying the composition of claim 1 to varicose veins of limbs in an amount sufficient to increase energy and activity levels.

17. A process comprising:
applying the composition of claim 1 topically on a human in an amount sufficient to promote regular bowel movements.

18. A process comprising:
applying a composition of DHEA topically in an amount sufficient to stimulate the transcription of youth-related genes.
